(19) United States
(12) Patent Application Publication
(10) Pub. No.: US 2020/0354736 A1
(43) Pub. Date: Nov. 12, 2020

(54) Modified plants and methods for reducing cell wall methylation and recalcitrance

(71) Applicant: The Regents of the University of California, Oakland, CA (US)

(72) Inventors: Dominique Loque, Vernier (CH); Aymerick G. Eudes, Emeryville, CA (US)

(73) Assignee: The Regents of the University of California, Oakland, CA (US)

(21) Appl. No.: 16/746,635

(22) Filed: Jan. 17, 2020

Related U.S. Application Data
(63) Continuation-in-part of application No. PCT/US2018/042562, filed on Jul. 17, 2018.
(60) Provisional application No. 62/533,621, filed on Jul. 17, 2017.

(51) Int. Cl. C12N 15/82; C12N 9/14; C13K 1/02

(52) U.S. Cl. CPC C12N 15/8255; C12N 9/14; C13K 1/02; C12N 15/8223; C12Y 303/01002

(57) ABSTRACT

The present invention provides for a genetically modified plant or plant cell comprising a nucleic acid encoding a S-adenosylmethionine hydrolase (AdoMetase) operatively linked to a tissue-specific secondary wall promoter such that there is a specific increased expression of AdoMetase in a secondary cell wall synthesizing tissue when compared secondary cell wall promoter expression compared to corresponding unmodified plant cells.

### RELATED PATENT APPLICATIONS

The application claims priority as a continuation-in-part application to International Patent Application PCT/US2018/42562, filed Jul. 17, 2018, which claims priority to U.S. Provisional Patent Application Ser. No. 62/533,621, filed Jul. 17, 2017, which are herein incorporated by reference in their entireties.

### STATEMENT OF GOVERNMENTAL SUPPORT

This invention was made with government support under Contract No. DE-AC02-05CH11231 awarded by the United States Department of Energy. The government has certain rights in the invention.

### FIELD OF THE INVENTION

This invention relates generally to modifying biomass by reducing cell wall methylation and recalcitrance.

### BACKGROUND OF THE INVENTION

Lignin is a phenolic polymer produced by oxidative polymerization of methylated hydroxycinnamyl alcohols (or monolignols) synthesized from phenylalanine (FIG. 1A). Guaiacyl (G) units are derived from coniferyl alcohol, which contains one methyl group, and syringyl (S) units are derived from dimethylated sinapyl alcohol (FIG. 1A). G and S units are the most common lignin monomers in angiosperms, whereas p-hydroxyphenyl (H) units derived from the polymerization of non-methylated p-coumaryl alcohol are typically less abundant (Boerjan et al., 2003). The enzymatic saccharification of lignocellulosic biomass for the production of fermentable sugars is negatively impacted by the presence of lignin, and consequently several strategies have been suggested to overcome lignin recalcitrance and to control its spatiotemporal deposition in bioenergy crops (Chen and Dixon, 2007; Eudes et al., 2014; Mottiar et al., 2016).

S-adenosylmethionine (AdoMet) is a key intermediate in one-carbon metabolism which serves as a universal methyl-group donor for the methylation of a large number of metabolites (Hanson and Roje, 2001). In addition to being used as a substrate by AdoMet-utilizing methyltransferases, it also acts as a precursor in the synthesis of polyamines, nicotianamine, phytosiderophores, 5′-deoxyadenosyl radicals, and ethylene (Roje, 2006). As part of the methionine salvage cycle (or Yang cycle), AdoMet is synthesized from methionine (Met) by AdoMet synthetase (FIG. 1B) (Albers, 2009).

The synthesis of lignin in tissues producing secondary cell walls (SCWs) is associated with a massive demand for one-carbon units and requires large supply and efficient recycling of AdoMet (Hanson and Roje, 2001; Amthor, 2003). The lignin biosynthetic pathway contains two enzymes that consume AdoMet for transmethylation reactions: Caffeoyl CoA O-methyltransferase (CCoAOMT) is involved in the first methylation step required for the production of G units, and caffeic acid O-methyltransferase (COMT) performs the second methylation step needed for the synthesis of S units (FIG. 1A) (Osakabe et al., 1999; Li et al., 2000; Zhong et al., 2001; Guo et al., 2001). Moreover, SCWs contain the hemicellulose 4-O-methylglucuronoxylan (GX), which also requires an important supply of AdoMet for synthesis (Scheller and Ulvskov, 2010). In particular, in Arabidopsis, three DUF579 domain-containing methyltransferases act redundantly for the 4-O-methylation of glucuronic acid (GlcA) side chains on GX (Urbanowicz et al., 2012; Lee et al., 2012; Yuan et al., 2014).

The importance of AdoMet as a methyl donor for lignin biosynthesis has been illustrated by several mutant studies in Arabidopsis and maize. For example, mutation in one of the AdoMet synthetases (AdoMetS3) in Arabidopsis results in concomitant reductions of AdoMet synthetase activity, AdoMet pools, and lignin content (Shen et al., 2002). Recently, it was shown that mutations in genes responsible for the synthesis of 5-methyltetrahydrofolate, which is used as methyl donor by Met synthase for the production of Met from homocysteine, leads to reductions of lignin content in maize and Arabidopsis (Tang et al., 2014; Li et al., 2015; Srivastava et al., 2015). These mutations affect methylenetetrahydrofolate reductase (MTHFR) or folylpolyglutamate synthase (FPGS), and reductions in both pools of AdoMet and its precursor Met were measured in the Arabidopsis fpgs mutant (Srivastava et al., 2011; 2015). Importantly, the Arabidopsis fpgs mutant is affected in a FPTGS isoform preferentially expressed in vascular tissues and does not show any defects in above ground biomass yield (Srivastava et al., 2015).

### SUMMARY OF THE INVENTION

The present invention provides a genetically modified plant or plant cell comprising a nucleic acid encoding an S-adenosylmethionine hydrolase (AdoMetase) operatively linked to a tissue-specific secondary wall promoter such that there is a specific increased expression of AdoMetase in a secondary cell wall synthesizing tissue when compared secondary cell wall promoter expression compared to corresponding unmodified plant cells.

As a result of the expression of AdoMetase in the genetically modified plant or plant cell, there is an increase in the H-units (see FIG. 1A) and/or β-O-4 linkages formed by the genetically modified plant or plant cell. An increase of H-units and/or β-O-4 linkages in the cell wall reduces the amount of lignin in the cell wall which in turn reduces the recalcitrance of the cell wall to deconstruction, including pretreatment.

The increased expression of AdoMetase results, such as an increased expression of AdoMetase in the secondary cell wall, in a reduction of S-Adenosylmethionine, which is a cofactor of methyltransferase. The invention targets the cofactor of methyltransferases specifically in tissues where they are involved in lignin synthesis rather than targeting directly these methyltransferases in the entire plant. Tissue-specific expression reduces the risk of any potential undesired effects caused by increased AdoMetase expression in the entire plant. The genetically modified plant or plant cell have a lower lignin content, due to an increase of less recalcitrant H-units and β-O-4 linkages, compared to corresponding unmodified plant or plant cell.

In some embodiments, the AdoMetase is heterologous to the genetically modified plant or plant cell and/or the tissue-specific secondary wall promoter. In some embodiments, the AdoMetase is coliphage T3 AdoMetase.

The amino acid sequence of coliphage T3 AdoMetase is:

```
(SEQ ID NO: 1)
                10 20 30 40
MIFTKEPAHV FYVLVSAFRS NLCDEVNMSR HRHMVSTLRA
                50 60 70 80
APGLYGSVES TDLTGCYREA ISSAPTEEKT VRVRCKDKAQ
                90 100 110 120
ALNVARLACN EWEQDCVLVY KSQTHTAGLV YAKGIDGYKA
              130 140 150
ERLPGSFQEV PKGAPLQGCF TIDEFGRRWQ VQ
```

In some embodiments, the tissue-specific secondary wall promoter is an IRX1, IRX3, IRX5, IRX8, IRX9, IRX14, IRX7, IRX10, GAUT13, GAUT14, or CESA4 promoter.

In some embodiments, the genetically modified plant or plant cell further comprises a nucleic acid encoding a transcription factor that regulates expression of the AdoMetase operably linked to the tissue-specific secondary wall promoter. For example, the transcription factor may be NAC secondary wall-thickening promoting factor 1 (NST1), NST2, NST3, secondary wall-associated NAC domain protein 2 (SND2), SND3, MYB domain protein 103 (MYB103), MBY85, MYB46, MYB83, MYB58, or MYB63.

Suitable tissue-specific secondary wall promoters, and other transcription factors, promoters, regulatory systems, and the like, suitable for this present invention are taught in U.S. Patent Application Pub. Nos. 2014/0298539, 2015/0051376, and 2016/0017355.

The present invention provides for a cell wall, or biomass obtained from the genetically modified plant of the present invention.

In some embodiments, the genetically modified plant or plant cell is selected from the group consisting of Arabidopsis, Camelina, flax, poplar, eucalyptus, rice, corn, switchgrass, sorghum, millet, miscanthus, sugarcane, pine, alfalfa, wheat, soy, barley, turfgrass, tobacco, hemp, bamboo, rape, sunflower, willow, and Brachypodium.

The present invention also provides for a method to produce cell wall modified with an increased amount of H-units and/or and β-O-4 linkages, comprising: (a) providing a genetically modified plant of the present invention, (b) growing or culturing the genetically modified plant under conditions in which the AdoMetase is expressed in a secondary cell wall synthesizing tissue, (c) optionally pretreating a biomass formed by the genetically modified plant to form a pretreated biomass, and (d) saccharifying the pretreated biomass to produce one or more sugars.

In some embodiments, the pretreating comprises contacting the biomass with a dilute alkaline solution.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1A. Simplified lignin biosynthetic pathway. Enzymatic steps consuming AdoMet in the lignin biosynthetic pathway are catalyzed by CCoAOMT and COMT. Abbreviations: ACC, 1-aminocyclopropanecarboxylate; AdoHCY, S-adenosylhomocysteine; AdoMet, S-adenosylmethionine; CCoAOMT, caffeoyl CoA O-methyltransferase; COMT, caffeic acid O-methyltransferase; CY, cystathionine; dcAdoMet, decarboxylated AdoMet; DHKMP, 1,2-dihydroxy-3-keto-5-methylthiopentene; DKPP, 2,3-diketo-5-methylthiopentyl-1-phosphate; ETHY, ethylene; HCY, homocysteine; HKMPP, 2-hydroxy-3-keto-5-methylthiopentenyl-1-phosphate; HS, homoserine; KMTB, α-ketomethylthiobutyrate; Met, methionine; MTA, methylthioadenosine; MTR, 5-methylthioribose; MTRP, 5-methylthioribose-1-phosphate; MTRuP, methylthioribulose-1-phosphate; NA, nicotianamide; PHS, β-phosphohomoserine; SPD, spermidine; THR, threonine. a, NA synthase; b, AdoMet decarboxylase; c, SPD synthase; d, ACC synthase; e, ACC oxidase.

FIG. 1B. The methionine salvage cycle (or Yang cycle). The metabolic shunt mediated by AdoMetase in the Yang cycle is shown in red. Abbreviations: ACC, 1-aminocyclopropanecarboxylate; AdoHCY, S-adenosylhomocysteine; AdoMet, S-adenosylmethionine; CCoAOMT, caffeoyl CoA O-methyltransferase; COMT, caffeic acid O-methyltransferase; CY, cystathionine; dcAdoMet, decarboxylated AdoMet; DHKMP, 1,2-dihydroxy-3-keto-5-methylthiopentene; DKPP, 2,3-diketo-5-methylthiopentyl-1-phosphate; ETHY, ethylene; HCY, homocysteine; HKMPP, 2-hydroxy-3-keto-5-methylthiopentenyl-1-phosphate; HS, homoserine; KMTB, α-ketomethylthiobutyrate; Met, methionine; MTA, methylthioadenosine; MTR, 5-methylthioribose; MTRP, 5-methylthioribose-1-phosphate; MTRuP, methylthioribulose-1-phosphate; NA, nicotianamide; PHS, O-phosphohomoserine; SPD, spermidine; THR, threonine. a, NA synthase; b, AdoMet decarboxylase; c, SPD synthase; d, ACC synthase; e, ACC oxidase; \textcircled{2}, MTA nucleosidase; \textcircled{3}, MTR kinase; \textcircled{4}, MTRP isomerase; \textcircled{5}, MTRuP dehydratase; \textcircled{6}, DKPP enolase; \textcircled{7}, HKMPP phosphatase, DHKMP dioxygenase; \textcircled{8}, KMTB aminotransferase. [symbol] HS kinase; [symbol] , CY synthase; [symbol] , CY β-lyase; [symbol] , Met synthase; [symbol] , Thr synthase.

FIG. 2A. AdoMetase expression in pAtIRX5::AdoMetase lines and phenotype. AdoMetase transcripts were detected by RT-PCR using stem mRNA from three independent 5-week-old T3 homozygous pAtIRX5::AdoMetase (AdoMetase) transformants. cDNA synthesized from stem mRNA of wild-type plants were used as a negative control. Tub8-specific primers were used to assess cDNA quality for each sample.

FIG. 2B. AdoMetase expression in pAtIRX5::AdoMetase lines and phenotype. Comparison of the growth and development of wild-type and pAtIRX5::AdoMetase (AdoMetase) lines at different stages. Upper panel: 3-week-old rosette; Middle panel: 5-week-old flowering stage; Bottom panel: 8-week-old senescing stage.

FIG. 3. Lignin content in senesced mature stems from wild-type and pAtIRX5::AdoMetase (AdoMetase) lines. Values are means±SE from four biological replicates (n=4). Asterisks indicate significant differences from the wild-type using the unpaired Student's t-test (*P<0.01).

FIG. 4A. Lignin composition and interunit linkages in senesced mature stems from wild-type and pAtIRX5::AdoMetase (AdoMetase) lines. The aromatic region of partial short-range ¹³C-¹H (HSQC) spectra of cell wall material are shown. Lignin monomer ratios and integration values for the α-C/H correlation peaks from the major lignin interunit structures are provided on the figures.

FIG. 4B. Lignin composition and interunit linkages in senesced mature stems from wild-type and pAtIRX5::AdoMetase (AdoMetase) lines. The aliphatic region of partial short-range ¹³C-¹H (HSQC) spectra of cell wall material are shown. Lignin monomer ratios and integration values for the α-C/H correlation peaks from the major lignin interunit structures are provided on the figures.

FIG. 5. Saccharification of biomass from mature senesced stems of wild-type and pAtIRX5::AdoMetase (AdoMetase) lines. Values represent the amounts of sugars released from biomass after a dilute alkaline pretreatment and 48-h enzymatic digestion with cellulase cocktail (1% w/w). Values are means±SE of six biological replicates (n=6). Asterisks indicate significant differences from the wild-type using the unpaired Student's t-test (*P<0.001).

FIG. 6. PCR results confirming the transfer of the AdoMetase gene into poplar lines constructed.

FIG. 7. Poplar plants engineered to express the AdoMetase gene grown to a height of about 3 cm.

### DETAILED DESCRIPTION OF THE INVENTION

The foregoing aspects and others will be readily appreciated by the skilled artisan from the following description of illustrative embodiments when read in conjunction with the accompanying drawings.

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” refers to a value including 10% more than the stated value and 10% less than the stated value.

In some embodiments, the saccharification of biomass grown or cultured from the genetically modified plants or plant cells of the present invention produces at least about 5%, 10%, 15%, 20%, 25%, or 30% more sugars when compared to the saccharification of biomass grown or cultured from corresponding unmodified plants or plant cells.

In some embodiments, the cell wall of the genetically modified plants or plant cells of the present invention having at least about 50%, 100%, 150%, 200%, 250%, or 300% more H-units when compared to the cell wall of biomass grown or cultured from corresponding unmodified plants or plant cells. In some embodiments, the cell wall of the genetically modified plants or plant cells of the present invention having an about 2.5 to 2.8-fold increase of H-units when compared to the cell wall of biomass grown or cultured from corresponding unmodified plants or plant cells.

In some embodiments, the cell wall of the genetically modified plants or plant cells of the present invention having at least about 1%, 2%, 3%, 4%, 5%, or 6% more G-units when compared to the cell wall of biomass grown or cultured from corresponding unmodified plants or plant cells.

In some embodiments, the cell wall of the genetically modified plants or plant cells of the present invention having at least about 5%, 10%, 15%, 20%, 25%, or 30% fewer S-units when compared to the cell wall of biomass grown or cultured from corresponding unmodified plants or plant cells. In some embodiments, the cell wall of the genetically modified plants or plant cells of the present invention having an about 1.4-fold decrease of S-units when compared to the cell wall of biomass grown or cultured from corresponding unmodified plants or plant cells.

### Example 1

### Expression of S-Adenosylmethionine Hydrolase in Tissues Synthesizing Secondary Cell Walls Alters Specific Methylated Cell Wall Fractions and Improves Biomass Digestibility

Plant biomass is a large source of fermentable sugars for the synthesis of bioproducts using engineered microbes. These sugars are stored as cell wall polymers, mainly cellulose and hemicellulose, and are embedded with lignin, which makes their enzymatic hydrolysis challenging. One of the strategies to reduce cell wall recalcitrance is the modification of lignin content and composition. Lignin is a phenolic polymer of methylated aromatic alcohols and its synthesis in tissues developing secondary cell walls is a significant sink for the consumption of the methyl donor S-adenosylmethionine (AdoMet). In this study, we demonstrate in Arabidopsis stems that targeted expression of S-adenosylmethionine hydrolase (AdoMetase, E. C. 3.3.1.2) in secondary cell-wall synthesizing tissues reduces the AdoMet pool and impacts lignin content and composition. In particular, both NMR analysis and pyrolysis gas chromatography mass spectrometry of lignin in engineered biomass showed relative enrichment of non-methylated p-hydroxycinnamyl (H) units and a reduction of dimethylated syringyl (S) units. This indicates a lower degree of methylation compared to that in wild-type lignin. Quantification of cell wall-bound hydroxycinnamates revealed a reduction of ferulate in AdoMetase transgenic lines. Biomass from transgenic lines, in contrast to that in control plants, exhibits an enrichment of glucose content and a reduction in the degree of hemicellulose glucuronoxylan methylation. We also show that these modifications resulted in a reduction of cell wall recalcitrance, because sugar yield generated by enzymatic biomass saccharification was greater than that of wild type plants. Considering that transgenic plants show no important diminution of biomass yields, and that heterologous expression of AdoMetase protein can be spatiotemporally optimized, this novel approach provides a valuable option for the improvement of lignocellulosic biomass feedstock.

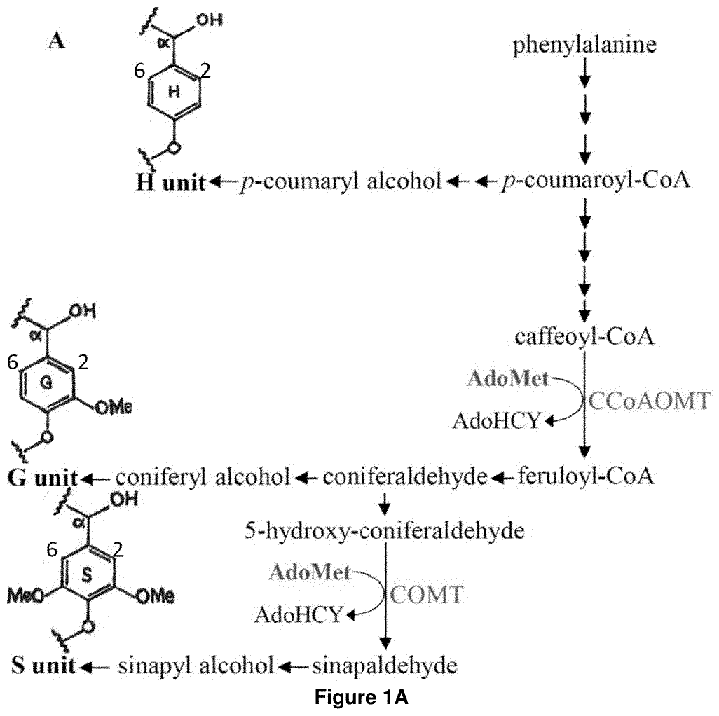

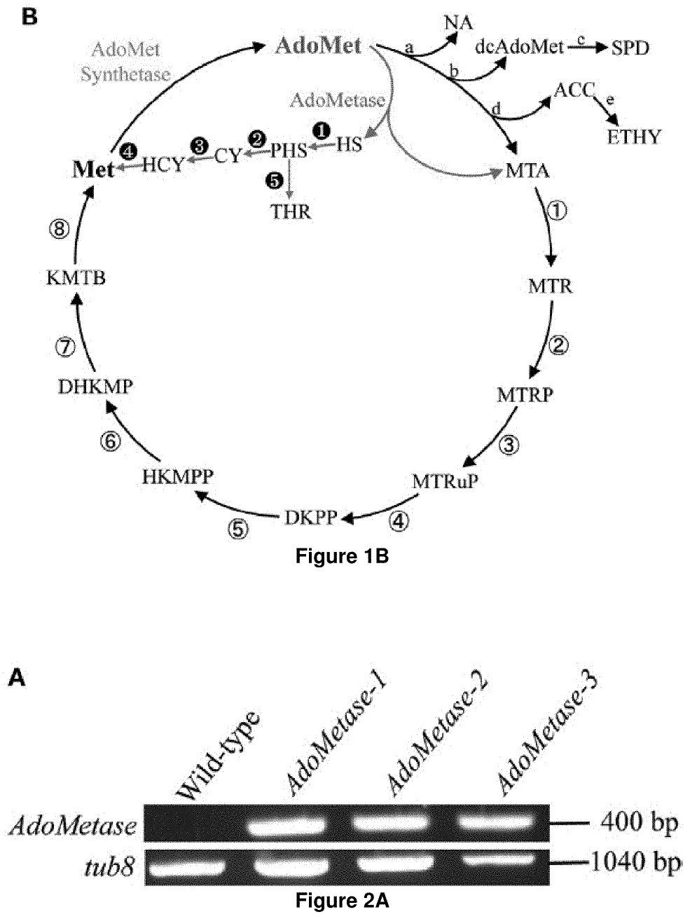

In this study, we evaluated in Arabidopsis the impact of expressing S-adenosylmethionine hydrolase (AdoMetase) in tissues producing SCWs. The AdoMetase gene has been cloned from the coliphage T3 (Hughes et al., 1987) and its product hydrolyzes AdoMet into homoserine and methylthioadenosine, which creates a metabolic shunt within the Yang cycle (FIG. 1B). Previous genetic engineering studies have demonstrated the efficacy of expressing AdoMetase stage-specifically in climacteric fruits to reduce ethylene production from AdoMet and slow the ripening process (Clendennen et al., 1999; Good et al., 1994; Mathews, 1995). We used in this study the promoter of a SCW cellulose synthase (pAtIRX5) to drive the expression of AdoMetase in stem interfascicular fibers and xylem vessels in which the biosynthesis of both lignin and GX requires significant amounts of AdoMet. Focusing our analyses on the main SCW components, we demonstrate that targeting the expression of the AdoMetase protein in SCW-producing tissues reduces the content of lignin and its degree of methylation, presumably by affecting simultaneously both methylation steps of the lignin biosynthetic pathway. We also show that biomass from engineered plants is characterized by an enrichment in glucose content, reduction of O-methylated GlcA residues on GX polymer, and lower amount of cell wall-bound ferulate. Although the transgenic plants show a reduction in stem size, their biomass yields are similar to those of wild-type plants, while their release of sugars from biomass upon enzymatic treatment is enhanced.

### Material and Methods

### Plant Material and Growth Conditions

Arabidopsis thaliana (ecotype Columbia, Col-0) seeds were germinated directly on soil. Growing conditions were 150 μmol/m²/s, 22° C., 60% humidity and 10 h of light per day. Selection of T2 and identification of T3 homozygous transgenic plants was made on Murashige and Skoog vitamin medium (PhytoTechnology Laboratories, Shawnee Mission, Kans.), supplemented with 1% sucrose, 1.5% agar, and 25 μg/mL hygromycin.

pAtIRX5::AdoMetase Construct and Plant Transformation

To generate the binary vector pA6-pAtIRX5::AdoMetase, the pAtIRX5 promoter described in Eudes et al. (2012) was released from pCR™-Blunt vector (Life Technologies, Foster City, Calif., USA) using KpnI/NheI restriction enzymes and ligated into the pA6-GW binary vector harboring a gateway cloning cassette (Yang et al., 2013) and digested with KpnI and AvrII (NheI compatible site) restriction enzymes to produce the pA6-pAtIRX5-GW binary vector. A nucleotide sequence encoding AdoMetase from the enterobacteria phage T3 (UniProtKB/Swiss-Prot accession number P07693.1) flanked with the Gateway attB1 (5′-end) and attB2 (3′-end) recombination sites was synthesized for expression in Arabidopsis (GenScript, Piscatway, N.J.) and cloned into the Gateway pDONR221-P1P2 entry vector by BP recombination (Life Technologies, Foster City, Calif., USA). An entry clone was LR recombined with the pA6-pAtIRX5-GW vector to generate the pA6-pAtIRX5::AdoMetase construct. The construct was introduced into wild-type Arabidopsis plants (ecotype Col0) via Agrobacterium tumefaciens-mediated transformation (Bechtold and Pelletier, 1998).

### RNA Extraction and RT-PCR

Total RNA (1 μg) was extracted from stems of 5-week-old wild-type and T3 homozygous transgenic lines using the Plant RNeasy extraction kit (Qiagen, Valencia, Calif.) and reverse-transcribed using the Transcriptor First Strand cDNA Synthesis Kit (Roche Applied Science, Indianapolis, Ind.). The cDNA preparations obtained were quality-controlled using the tub8-specific oligonucleotides Tub8-fw and Tub8-rv (Table 6), and the AdoMetase transcripts were detected using the oligonucleotides AdoMetase-fw and AdoMetase-rv (Table 6).

TABLE 6
Oligonucleotides used in Example 1

| Primer name | Sequence (5′-3′) |
|---|---|
| Tub8-fw | GGGCTAAAGGACACTACACTG |
| | (SEQ ID NO: 2) |
| Tub8-rv | CCTCCTGCACTTCCACTTCGTCTTC |
| | (SEQ ID NO: 3) |
| AdoMetase-fw | CCAATCTTTGCGATGAGGTTAATATG |
| | (SEQ ID NO: 4) |
| AdoMetase-rv | GTCCTGAACTTGCCACCTTCTTC |
| | (SEQ ID NO: 5) |

### Metabolites Extraction

Arabidopsis stems of 5-week-old wild-type and T3 homozygous pAtIRX::AdoMetase lines were collected in liquid nitrogen and stored at −80° C. until further utilization. Collected stems were pulverized in liquid nitrogen and metabolites were extracted as previously described (Van de Poel et al., 2010): 100-200 mg of frozen stem powder was homogenized with 1 ml of trichloroacetic acid (5% w/v) and mixed (1,400 rpm) for 15 min at 4° C. Extracts were cleared by centrifugation (10 min, 20,000×g, at 4° C.) and filtered using Amicon Ultra centrifugal filters (3,000 Da MW cutoff regenerated cellulose membrane; EMD Millipore, Billerica, Mass.). Filtered extracts were kept at −20° C. until LC-MS analysis.

### Cell-Wall Bound Hydroxycinnamates Extraction

The biomass from senesced wild-type plants and T3 homozygous pAtIRX::AdoMetase lines was used to measure cell-wall bound ferulate and p-coumarate as previously described (Eudes et al., 2015). Extracted biomass (10 mg) was mixed with 500 μL of 2 M NaOH and shaken at 1,400 rpm for 24 h at 30° C. The mixture was acidified with 100 μL of concentrated HCl (12N), and subjected to three ethyl acetate partitioning steps. Ethyl acetate fractions were pooled, dried in vacuo, and suspended in 50% (v/v) methanol-water prior to LC-MS analysis.

### Biomass Compositional Analysis

The biomass from senesced wild-type plants and T3 homozygous pAtIRX5::AdoMetase lines was used for analysis. Biomass was extracted sequentially by sonication (20 min) with 80% (v/v) ethanol-water (three times), 100% acetone (one time), chloroform-methanol (1:1, v/v, one time) and 100% acetone (one time). The standard NREL protocol consisting of a two-step acid hydrolysis of biomass was used to measure lignin content and determine monosaccharide composition (Sluiter et al. 2008). Hydrolysis of biomass with trifluoroacetic acid was performed as previously described (Eudes et al., 2012) for the release of glucose residues that are not polymerized into crystalline cellulose. The chemical composition of lignin was analyzed by pyrolysis-gas chromatography (GC)/mass spectrometry (MS) using a previously described method (Eudes et al., 2012). Lignin pyrolysis products were identified by comparing their mass spectra with those of the NIST library and those previously reported (Del Río and Gutiérrez, 2006; Ralph and Hatfield, 1991).

### LC-MS Analysis

High-performance liquid chromatography (HPLC) mobile phases were composed of HPLC grade solvents. AdoMet, homoserine, methylthioadenosine, homocysteine, methionine, and threonine were analyzed using HPLC, electrospray ionization (ESI), and time-of-flight (TOF) mass spectrometry (MS) as previously described in Bokinsky et al. (2013). Ferulate and p-coumarate were analyzed using HPLC-ESI-TOF-MS as previously described (Eudes et al., 2013). 4-O-MeGlcA from biomass hydrolyzates was analyzed and quantified by HPLC-ESI-TOF-MS. The separation of MeGlcA was conducted on a Carbomix H-NP5 column with 8% cross linkage (150 mm length, 4.6 mm internal diameter, and 5 μm particle size; Sepax Technologies, DE, USA) using an Agilent Technologies 1200 Series Rapid Resolution HPLC system. The temperature of the sample tray was maintained at 6° C. by an Agilent FC/ALS Thermostat. The column compartment was set to 50° C. A sample injection volume of 5 μL was used. Metabolites were eluted isocratically with a mobile phase composed of 0.1% formic acid in water. A flow rate of 0.25 mL/min was used throughout. The HPLC system was coupled to an Agilent Technologies 6210 TOF mass spectrometer by a 1/4 post-column split. Contact between both instrument set-ups was established by a LAN card to trigger the MS into operation upon the initiation of a run cycle from the MassHunter workstation (Agilent Technologies, CA, USA). Nitrogen was used as both the nebulizing and drying gas to facilitate the production of gas-phase ions. Drying and nebulizing gases were set to 11 L/min and 30 psi, respectively, and a drying gas temperature of 330° C. was used throughout. ESI was conducted in the negative ion mode and a capillary voltage of −3,500 V was utilized. MS experiments were carried out in the full scan mode, at 0.86 spectra/second, for the detection of [M-H]⁻ ions. The instrument was tuned for a range of 50 to 1700 m/z and ions were acquired from 100 to 1000 m/z. Prior to LC-TOF MS analysis, the TOF MS was calibrated via an ESI-L low concentration tuning mix (Agilent Technologies, CA, USA). Data acquisition and processing were performed by the MassHunter software package. A 4-O-MeGlcA authentic standard was obtained from LC Scientific Inc. (Concord, Canada). All metabolites were quantified via calibration curves of authentic standard compounds for which the R² coefficients were >0.99.

### HPAEC-PAD Analysis

Except for the quantification of 4-O-MeGlcA for which LC-MS analysis was used because of higher sensitivity (see method section above), the monosaccharide composition of hydrolyzed biomass was determined by HPAEC-PAD. Measurements of fucose, rhamnose, arabinose, galactose, glucose, galacturonic acid and glucuronic acid contents were conducted as previously described (Eudes et al., 2012). Because this method cannot separate xylose from mannose, a different HPAEC-PAD method was used for the separation and measurement of these two monosaccharides: The chromatography was performed on a PA20 column (Dionex, Sunnyvale, Calif.) at a flow rate of 0.4 ml min⁻¹ and the column oven set at 30° C. Before injection of each sample (20 μl) the column was washed with 200 mM NaOH for 10 min and equilibrated with 4 mM NaOH for 5 min. The elution program was as follows: 6 min with 4 mM NaOH, ramp down to 1 mM NaOH for 2 min; 11 min at 1 mM NaOH, ramp to 450 mM NaOH for 6 seconds; then 450 mM NaOH for 18 min. Monosaccharides were detected using a pulsed amperometric detector (gold electrode) set on waveform A according to manufacturer's instructions. A calibration curve of monosaccharide standards that includes L-fucose, L-rhamnose, L-arabinose, D-galactose, D-glucose, D-xylose, D-mannose, D-galacturonic acid and D-glucuronic acid (Sigma-Aldrich, St Louis, Mo.) was run for verification of response factors.

2D ¹³C-¹H Heteronuclear Single Quantum Coherence (HSQC) NMR Spectroscopy

Stem material from wild-type and pAtIRX5::AdoMetase lines was extracted and ball milled as previously described (Kim and Ralph, 2010; Mansfield et al., 2012). The gels were formed using DMSO-d₆/pyridine-d₅ (4:1) and sonicated until homogenous in a Branson 2510 table-top cleaner (Branson Ultrasonic Corporation, Danbury, Conn.). The homogeneous solutions were transferred to NMR tubes. HSQC spectra were acquired at 25° C. using a Bruker Avance-600 MHz instrument equipped with a 5 mm inverse-gradient ¹H/¹³C cryoprobe using a hsqcetgpsisp2.2 pulse program (ns=400, ds=16, number of increments=256, d₁=1.0 s) (Heikkinen et al., 2003). Chemical shifts were referenced to the central DMSO peak (δ_{C}/δ_{H} 39.5/2.5 ppm). Assignment of the HSQC spectra was described elsewhere (Kim and Ralph, 2010; Yelle et al., 2008). A semi-quantitative analysis of the volume integrals of the HSQC correlation peaks was performed using Bruker's Topspin 3.1 (Windows) processing software. A Guassian apodization in F₂ (LB=−0.50, GB=0.001) and squared cosine-bell in F₁ (LB=−0.10, GB=0.001) were applied prior to 2D Fourier transformation.

### Cell Wall Pretreatments and Saccharification

Ball-milled senesced stems (10 mg) were mixed with 340 μL of NaOH (0.25%, w/v), shaken at 1,400 rpm (30° C., 30 min), and autoclaved at 120° C. for 1 h. Saccharification was initiated by adding 650 μL of 100 mM sodium citrate buffer pH 5 containing 80 μg/mL tetracycline and 1% w/w Cellic CTec2 cellulase (Novozymes, Davis, Calif.). After 48 h of incubation at 50° C. with shaking (800 rpm), samples were centrifuged (20,000×g, 3 min) and 10 μL of the supernatant was collected for measurement of reducing sugars using the 3,5-dinitrosalicylic acid assay and glucose solutions as standards (Miller, 1959).

### Results

### Expression of AdoMetase in Arabidopsis

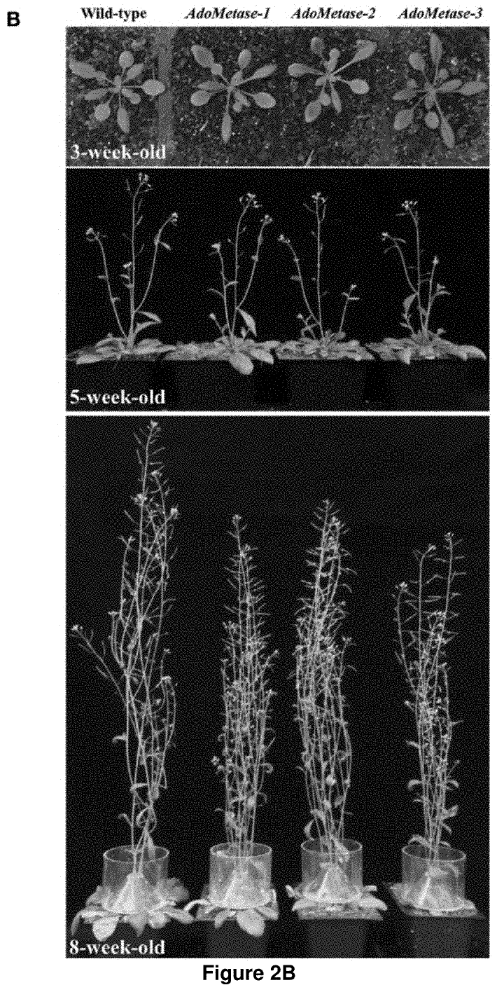

The promoter of the SCW cellulose synthase gene AtCesA4 (pAtIRX5), which is specifically active in interfascicular fibers and xylem vessels (Eudes et al., 2012), was selected to express specifically the AdoMetase protein in Arabidopsis stems. Reverse transcription PCR (RT-PCR) using mRNA from stems of three independent homozygous transformants confirmed AdoMetase expression (FIG. 2A). These lines are fertile, and, compared to wild-type, show no obvious growth defect phenotype nor a decrease in biomass yield of total stems, but exhibit a 12-20% height reduction of the main stem (FIG. 2B, Table 1).

TABLE 1
Height of the main inflorescence stem and total stem dry weight of senesced
mature wild-type and pAtIRX5::AdoMetase (AdoMetase) lines. Values are means ± SE from six
biological replicates (n = 6). Asterisks indicate significant differences from the wild-type using
the unpaired Student's t-test (*P < 0.05).

| Plant line | Height (cm) | Dry weight (mg) |
|---|---|---|
| Wild type | 62.3 ± 2.4 | 263.9 ± 21.4 |
| AdoMetase-1 | 53.8 ± 2.1* | 246.2 ± 26.3 |
| AdoMetase-2 | 55.1 ± 1.0* | 277.6 ± 20.3 |
| AdoMetase-3 | 50.2 ± 71.7* | 234.6 ± 38.7 |

Metabolite Analysis of pAtIRX5::AdoMetase Lines

Metabolites from stems of the pAtIRX5::AdoMetase lines were extracted and analyzed using LC-MS (Table 2). Compared to wild-type, AdoMet content was reduced by 38-51% in transgenic plants, and the AdoMetase activity products homoserine and methyltioadenosine were detected only in stems of pAtIRX5::AdoMetase lines. These results confirm the activity of AdoMetase in Arabidopsis stems and validate its use to reduce the AdoMet pool. Similarly, homocysteine was detected only in transgenic plants. The latter could be the result of higher homoserine conversion via O-phosphohomoserine and cystathionine (FIG. 1B), which is supported with higher amounts of threonine (3-fold) measured in stems of pAtIRX5::AdoMetase plants. Measurement of Met showed unchanged content in two lines and a 1.9-fold increase in the third one.

TABLE 2
Quantitative analysis of metabolites in stems from five-week-old wild-type and
pAtIRX5::AdoMetase (AdoMetase) lines. Values are means ± SE from six biological replicates (n =
6). nd, Not detected. Asterisks indicate significant differences from the wild-type using the
unpaired Student's t-test (*P < 0.005).

| Mean ± SE (nmole | Plant line | | | |
|---|---|---|---|---|
| g⁻¹ fresh weight) | Wild type | AdoMetase-1 | AdoMetase-2 | AdoMetase-3 |
| AdoMet | 18.5 ± 0.7 | 11.5 ± 1.1* | 9.5 ± 1.1* | 9.1 ± 0.7* |
| Homoserine | nd | 13.0 ± 2.7 | 12.5 ± 1.4 | 10.7 ± 1.3 |
| Methylthioadenosine | nd | 2.2 ± 0.2 | 2.2 ± 0.1 | 2.3 ± 0.1 |
| Homocysteine | nd | 25.2 ± 2.5 | 11.9 ± 3.0 | 20.9 ± 5.2 |
| Threonine | 551 ± 67 | 1628 ± 93* | 1681 ± 131* | 1634 ± 109* |
| Methionine | 25.7 ± 3.3 | 23.7 ± 1.3 | 24.7 ± 2.6 | 49.2 ± 5.6* |

Cell wall-bound p-coumarate and ferulate released from cell walls by mild alkaline hydrolysis were also analyzed using LC-MS (Table 3). The content of p-coumarate was unchanged in the pAtIRX5::AdoMetase lines whereas ferulate was reduced by 22-24%.

TABLE 3
Quantitative analysis of cell wall-bound ferulate and p-coumarate in stems from
senesced mature dried wild-type and pAtIRX5::AdoMetase (AdoMetase) lines. Values are means ±
SE from four biological replicates (n = 4). Asterisks indicate significant differences from the
wild-type using the unpaired Student's t-test (*P < 0.05).

| | Mean ± SE (μg g⁻¹ dry weight) | |
|---|---|---|
| Plant line | Ferulate | p-Coumarate |
| Wild type | 15.2 ± 0.7 | 23.9 ± 3.8 |
| AdoMetase-1 | 11.7 ± 0.8* | 23.7 ± 5.0 |
| AdoMetase-2 | 11.8 ± 0.8* | 24.6 ± 3.5 |
| AdoMetase-3 | 11.5 ± 0.6* | 23.3 ± 1.4 |

Lignin Content and Monomeric Composition in pAtIRX5::AdoMetase Lines

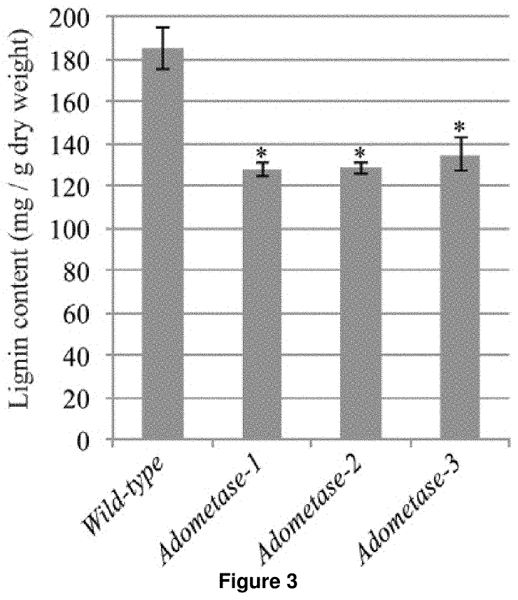

The Klason method was used to measure lignin content and revealed reductions ranging from 27-31% in stems of the pAtIRX5::AdoMetase lines compared to wild-type (FIG. 3). Cell wall material from stems of wild-type and pAtIRX5::AdoMetase lines was analyzed by pyrolysis-GC/MS for the determination of the lignin monomer composition. For each line, identification and relative quantification of the pyrolysis products derived from H, G, or S units allowed determination of H/G/S ratios (Table 4, Table 6). In transgenic plants, the relative amount of G units is unchanged, whereas that of H and S units is increased by 2.5-2.8-fold and reduced by ˜1.4-fold, respectively.

TABLE 4
Lignin monomeric composition in senesced mature stems from wild-type and
pAtIRX5::AdoMetase (AdoMetase) lines. Values are means ± SE from four biological replicates
(n = 4). Asterisks indicate significant differences from the wild-type using the unpaired Student's
t-test (*P < 0.005).

| | % H | % G | % S | S/G |
|---|---|---|---|---|
| Wild type | 3.2 ± 0.4 | 67.1 ± 1.2 | 29.7 ± 1.1 | 0.44 |
| AdoMetase-1 | 8.2 ± 0.3* | 70.7 ± 1.2 | 21.1 ± 1.1* | 0.30* |
| AdoMetase-2 | 9.1 ± 1.1* | 70.9 ± 1.1 | 20.0 ± 1.4* | 0.28* |
| AdoMetase-3 | 8.1 ± 0.8* | 71.1 ± 1.3 | 20.7 ± 1.5* | 0.29* |

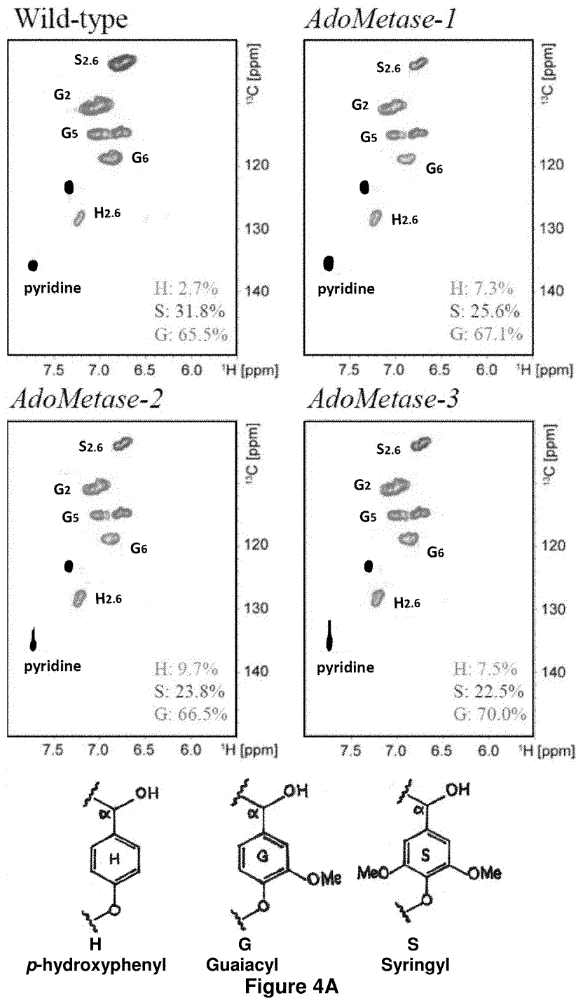

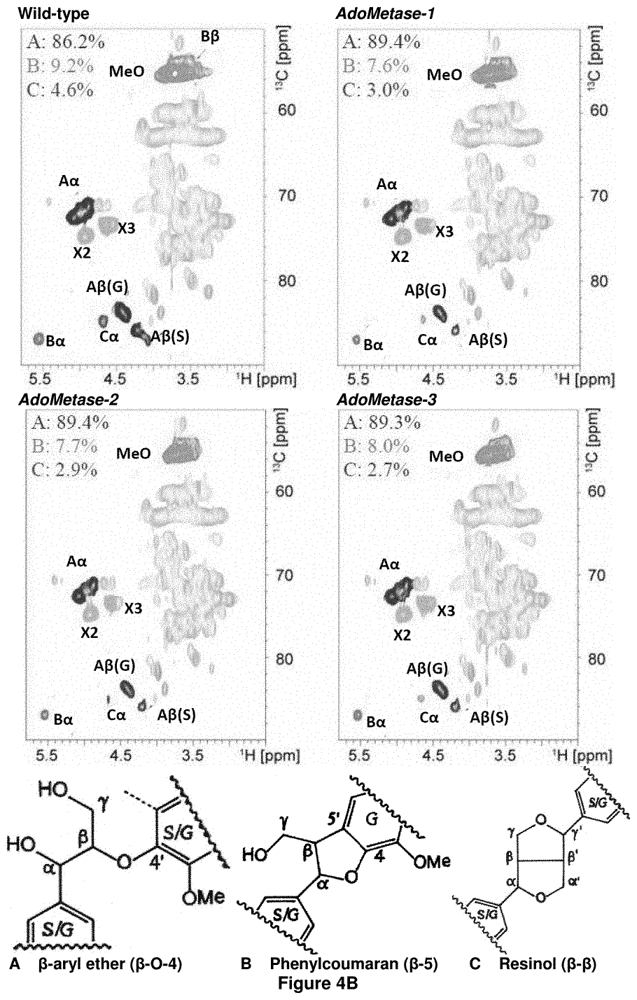

NMR (2D ¹³C-¹H-correlated, HSQC) spectra of cell wall material from wild-type and pAtIRX5::AdoMetase plants were also obtained to determine lignin composition and structure. Analysis of the aromatic region of the spectra confirmed the higher relative amount of H units in transgenics (7.3-9.7%) compared to wild-type (2.7%), as well as a reduction of S units (FIG. 4A). Moreover, analysis of the aliphatic region of the spectra indicated an increase of β-aryl ether (β-O-4) linkages and diminution of phenylcoumaran (β-5) and resinol (β-β) linkages in the lignin of transgenic plants (FIG. 4B).

Monosaccharide Composition in pAtIRX5::AdoMetase Lines

Monosaccharide composition was determined in mature senesced stems after sulfuric acid hydrolysis of total cell wall polysaccharides (Table 5). HPAEC-PAD and LC-MS analyses of cell wall hydrolysates showed that biomass from pAtIRX5::AdoMetase lines contains less 4-O-MeGlcA (−72%) but more non-methylated GlcA (+58-60%) as well as more glucose (+9-13%) and mannose (+24-36%). Considering that GX is the main source of 4-O-MeGlcA and GlcA residues in cell wall biomass of mature Arabidopsis stems, we conclude that the pAtIRX5::AdoMetase lines have a lower GX methylation degree (˜25-26% of total GlcA residues) compared to wild-type (˜75% of total GlcA residues). Moreover, higher amount of mannose along with an increase of glucose content measured in trifluoroacetic acid hydrolysates of cell wall residues from the transgenic lines could be explained by an enrichment in hemicellulosic glucomannan (Table 5).

TABLE 5
Chemical composition of total cell wall sugars in senesced mature dried stems
from wild-type and pAtIRX5::AdoMetase (AdoMetase) lines. “Glucose (TFA)” is the amount of
glucose released after trifluoroacetic acid hydrolysis of cell wall residues. Values are means ±
SE from three biological replicates (n = 3). Asterisks indicate significant differences from the
wild-type using the unpaired Student's t-test (*P < 0.05).

| | Mean ± SE (mg g⁻¹ dry weight) | | | |
|---|---|---|---|---|
| Sugar | Wild-type | AdoMetase-1 | AdoMetase-2 | AdoMetase-3 |
| Fucose | 3.1 ± 0.1 | 3.1 ± 0.0 | 3.1 ± 0.0 | 3.1 ± 0.0 |
| Rhamnose | 7.2 ± 0.2 | 6.6 ± 0.1 | 6.5 ± 0.2 | 6.4 ± 0.2 |
| Arabinose | 7.8 ± 0.1 | 8.1 ± 0.3 | 7.6 ± 0.3 | 7.9 ± 0.2 |
| Galactose | 13.2 ± 0.4 | 13.5 ± 0.4 | 13.3 ± 0.4 | 13.7 ± 0.3 |
| Mannose | 22.7 ± 1.8 | 35.3 ± 3.2* | 30.8 ± 3.2* | 29.8 ± 0.9* |
| Galacturonic acid | 61.9 ± 8.9 | 51.9 ± 6.9 | 55.6 ± 5.3 | 65.8 ± 1.1 |
| Xylose | 185.1 ± 12.7 | 220.8 ± 9.9 | 192.9 ± 10.0 | 184.2 ± 9.4 |
| Glucose | 376.4 ± 9.2 | 433.3 ± 13.4* | 422.2 ± 15.7* | 413.7 ± 13.7* |
| Glucose (TFA) | 10.4 ± 0.3 | 12.9 ± 0.2* | 14.3 ± 0.3* | 14.2 ± 0.3* |
| Glucuronic acid | 2.2 ± 0.1 | 5.2 ± 0.8* | 5.2 ± 0.8* | 5.6 ± 0.2* |
| 4-O-Methylglucuronic acid | 6.8 ± 0.8 | 1.9 ± 0.3* | 1.9 ± 0.1* | 1.9 ± 0.2* |

Saccharification Efficiency in pAtIRX5::AdoMetase Lines

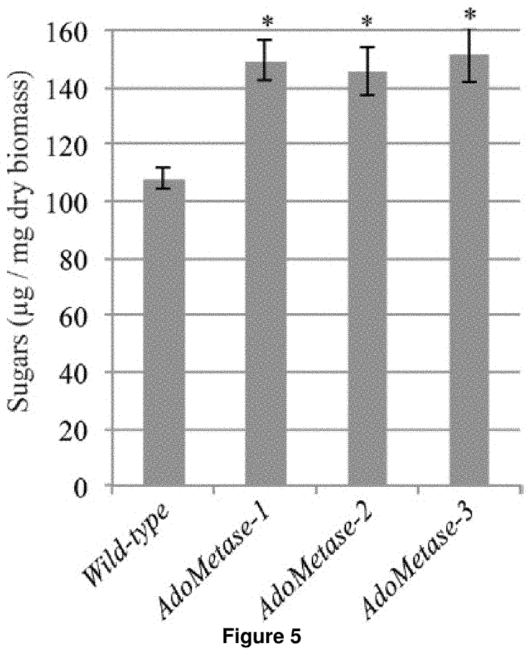

Saccharification assays after dilute alkaline pretreatment of stem material were conducted to evaluate the cell wall digestibility of the pAtIRX5::AdoMetase lines. As shown in FIG. 5, higher amount of sugars (+26-29%) were released from the biomass of the transgenic lines after 48 h enzymatic hydrolysis with commercial cellulase cocktail. These data demonstrate that cell wall biomass from the pAtIRX5::AdoMetase lines is less recalcitrant to cellulase digestion.

### Discussion

Cellulosic biomass contains 20-30% lignin, and more than 10% of carbon stored in the lignin is derived from AdoMet. In particular, G and S lignin units, which represent more than 95% of lignin units, consist of ten and eleven carbon skeletons, respectively, and harbor one or two methyl groups added by AdoMet-dependent methyltransferases (CCoAOMT and COMT). This makes lignin the major sink for AdoMet utilization in stem tissues developing SCWs. Here we show that expressing AdoMetase in these tissues reduces lignin content. AdoMetase cleaves AdoMet and generates homoserine and methylthioadenosine (FIG. 1B). Our metabolite analysis of plants expressing AdoMetase revealed a ˜50% reduction of AdoMet pools, whereas Met content was not reduced compared to wild-type (Table 2). These data suggest that AdoMet synthetase activity becomes a limiting factor and cannot compensate for AdoMetase activity to maintain AdoMet pools at the level of the wild-type. Furthermore, AdoMetase generates homoserine, which can potentially be recycled into Met and threonine via O-phosphohomoserine (FIG. 1B; Jander and Joshi, 2009). In the case of AdoMetase transgenic lines, such recycling could be occurring during Met synthesis since accumulation of homocysteine was observed, and likely takes place during the synthesis of threonine, as its content is increased threefold. For undetermined reasons, Met content was ˜twofold higher in one of the AdoMetase transgenic lines, which could be due to higher AdoMetase activity, more efficient recycling of homoserine into Met, or the consequence of higher activity of one or several enzymes of the Yang cycle (\textcircled{1} to \textcircled{8} on FIG. 1B).

In Arabidopsis, AdoMet content is not considered to be limiting for lignin biosynthesis (for S units in particular) and GX methylation since overexpression of ferulate 5-hydroxylase and of GX methyltransferase result in higher S-lignin units and a higher degree of GX methylation, respectively (Meyer et al., 1998; Yuan et al., 2014). Nevertheless, our data show that a ˜50% reduction of AdoMet content in Arabidopsis stems due to AdoMetase expression leads to reductions of lignin content and degree of GX methylation. In our analysis of cell wall monosaccharides, we observed in AdoMetase transgenic lines a reduction of 4-O-MeGlcA which is associated with an increase of GlcA and no change in xylose content, showing that reducing the degree of GX methylation degree does not affect GX content. This has been observed previously in GX methyltransferases mutants that display various degrees of GX methylation and yet showed no differences for the content of monosaccharides (Urbanowicz et al., 2012; Lee et al., 2012; Yuan et al., 2014). These results also suggest that the increase of glucose observed in cell walls of the AdoMetase transgenic lines would be a consequence of the reduction of lignin rather than the change in GX methylation. Both CCoAOMT and COMT are predicted to be located in the cytosol for the biosynthesis of lignin (Ruelland et al., 2003; Tanz et al., 2013), whereas GX methyltransferases are localized in the Golgi apparatus for the methylation of GlcA residues (Urbanowicz et al., 2012; Lee et al., 2012), which implies that AdoMetase affects AdoMet pools in both the cytosol and the Golgi apparatus.

Lignin content is reduced by ˜30% in the AdoMetase transgenic lines compared to that in wild-type plants, and the S/G ratio is decreased due to relative reduction of S units, indicating that synthesis of both G and S units is compromised. Moreover, lignin from the transgenic lines shows a relative increase of H units, which has been previously observed in lignins from plants and cell cultures affected in CCoAOMT activity (Do et al., 2008; Wagner et al., 2011). In AdoMetase transgenic plants, we also measured a lower amount of cell wall-bound ferulate, which is derived from the incorporation of feruloyl-CoA synthesized by CCoAOMT. These observations indicate that both of the methylation steps catalyzed by CCoAOMT and COMT in the lignin biosynthetic pathway are partially inhibited in transgenic lines. Nevertheless, CCoAOMT and COMT are possibly affected differently in AdoMetase-expressing lines, depending on their respective affinity for AdoMet and activity at lower intracellular AdoMet concentration. Furthermore, reduction of CCoAOMT and COMT activities has to be moderate in these lines since simultaneous disruption of both genes results in growth arrest at the seedling stage (Do et al., 2008).

We show that cell wall biomass from plants expressing AdoMetase is less recalcitrant to cellulose degradation, which probably results from changes in lignin content and structure since even a complete loss of GX methylation was shown not to affect glucose yields during saccharification (Yuan et al., 2014). Lignin is presumably less recalcitrant in the case of the AdoMetase transgenic lines because it contains higher amounts of the more labile β-O-4 linkages and fewer condensed C-C linkages. Moreover, an increase in the relative amount of H units typically affects the degree of polymerization of lignin and its extractability under alkaline treatment, which could promote biomass saccharification efficiency (Ziebell et al., 2010; Eudes et al., 2015).

Many metabolites other than lignin monomers require one-carbon units and AdoMet for biosynthesis, although in these cases the demand in SCW-producing tissues is considerably below that allocated for lignin biosynthesis (Hanson and Roje, 2001). Nevertheless, in stems of the AdoMetase transgenic lines, it would be interesting to assess the impact of AdoMetase expression on the content of metabolites derived from AdoMet such as thermospermine and ethylene, both of which have been found to be detrimental to differentiation of plant cells into vascular cells (Bollhöner et al., 2012; Takano et al., 2012). One hypothesis is to suggest that AdoMet levels are still sufficient to sustain the synthesis of these metabolites in stems of AdoMetase-expressing plants because AdoMetase is expressed under the control of the promoter of an SCW cellulose synthase gene (IRX5) that is active after xylem cell differentiation, when cells turn on their SCW synthesis machinery. In an observation similar to ours, mutation of FPGS1, which is preferentially expressed in vascular tissues of Arabidopsis, resulted in a 50% reduction of AdoMet content in stems, a 17% reduction in lignin content, and no loss of above-ground biomass (Srivastava et al., 2015).

In our view, expression of AdoMetase offers promise as a new approach for engineering bioenergy crops though modification of lignin. To optimize biomass improvement without affecting yields, such heterologous expression could be conducted with higher precision using different tissue-specific promoters that are also active at different SCW developmental stages.

### REFERENCES CITED

Albers, E. (2009). Metabolic characteristics and importance of the universal methionine salvage pathway recycling methionine from 5′-methylthioadenosine. IUBMB Life 61, 1132-1142.
Amthor, J. S. (2003). Efficiency of lignin biosynthesis: a quantitative analysis. Ann. Bot. 91, 673-695.
Bechtold, N. and Pelletier, G. (1998). In planta Agrobacterium-mediated transformation of adult Arabidopsis thaliana plants by vacuum infiltration. Methods in molecular biology (Clifton, N.J.) 82, 259-266.
Boerjan, W., Ralph, J. and Baucher, M. (2003). Lignin biosynthesis. Annu. Rev. Plant Biol. 54, 519-546.
Bokinsky, G., Baidoo, E. E., Akella, S., Burd, H., Weaver, D., Alonso-Gutierrez, J., Garcia-Martin, H., Lee, T. S. and Keasling, J. D. (2013). HipA-triggered growth arrest and β-lactam tolerance in Escherichia coli are mediated by RelA-dependent ppGpp synthesis. J. Bacteriol. 195, 3173-3182.
Bollhöner, B., Prestele, J. and Tuominen, H. (2012). Xylem cell death: emerging understanding of regulation and function. J. Exp. Bot. 63, 1081-1094.
Chen, F. and Dixon, R. A. (2007). Lignin modification improves fermentable sugar yields for biofuel production. Nat. Biotechnol. 25, 759-761.
Clendennen, S., Kellogg, J. A., Wolf, K. A., Matsumura, W., Peters, S., Vanwinkle, J. E., Copes, B., Pieper, M. and Kramer, M. G. (1999). Genetic engineering of cantaloupe to reduce ethylene biosynthesis and control ripening. In: Kanellis, A., Chang, C., Klee, H., Bleecker, A. B., Pech, J. C., Grierson, D. (Eds.) Biology and Biotechnology of the Plant Hormone Ethylene II. Kluwer Academic Publishers, Netherlands, pp. 371-379.
Del Río, J. C. and Gutiérrez, A. (2006). Chemical composition of abaca (Musa textilis) leaf fibers used for manufacturing of high quality paper pulps. J. Agric. Food Chem. 54, 4600-4610.
Do, C. T., Pollet, B., Thévenin, J., Sibout, R., Denoue, D., Barrière, Y., Lapierre, C. and Jouanin, L. (2007). Both caffeoyl Coenzyme A 3-O-methyltransferase 1 and caffeic acid O-methyltransferase 1 are involved in redundant functions for lignin, flavonoids and sinapoyl malate biosynthesis in Arabidopsis. Planta. 226, 1117-1129.
Eudes, A., George, A., Mukerjee, P., Kim, J. S., Pollet, B., Benke, P. I., Yang, F., Mitra, P., Sun, L., Cetinkol, O. P., Chabout, S., Mouille, G., Soubigou-Taconnat, L., Balzergue, S., Singh, S., Holmes, B. M., Mukhopadhyay, A., Keasling, J. D., Simmons, B. A., Lapierre, C., Ralph, J. and Loqué, D. (2012). Biosynthesis and incorporation of side-chain-truncated lignin monomers to reduce lignin polymerization and enhance saccharification. Plant Biotechnol. J. 10, 609-620.
Eudes, A., Juminaga, D., Baidoo, E. E., Collins, F. W., Keasling, J. D. and Loqué, D (2013). Production of hydroxycinnamoyl anthranilates from glucose in Escherichia coli. Microb. Cell Fact. 12, 62.
Eudes, A., Liang, Y., Mitra, P. and Loqud, D. (2014). Lignin bioengineering. Curr. Opin. Biotechnol. 26, 189-198.
Eudes, A., Sathitsuksanoh, N., Baidoo, E. E., George, A., Liang, Y., Yang, F., Singh, S., Keasling, J. D., Simmons, B. A. and Loqué, D. (2015). Expression of a bacterial 3-dehydroshikimate dehydratase reduces lignin content and improves biomass saccharification efficiency. Plant Biotechnol. J. 13, 1241-1250.
Good, X., Kellogg, J. A., Wagoner, W., Langhoff, D., Matsumura, W. and Bestwick, R. K. (1994). Reduced ethylene synthesis by transgenic tomatoes expressing S-adenosylmethionine hydrolase. Plant Mol. Biol. 26, 781-790.
Guo, D., Chen, F., Inoue, K., Blount, J. W. and Dixon R. A. (2001). Downregulation of caffeic acid 3-O-methyltransferase and caffeoyl CoA 3-O-methyltransferase in transgenic alfalfa. Impacts on lignin structure and implications for the biosynthesis of G and S lignin. Plant Cell 13, 73-88.
Hanson, A. D. and Roje, S. (2001). One-carbon metabolism in higher plants. Annu. Rev. Plant. Biol. 52, 119-137.
Heikkinen, S., Toikka, M. M., Karhunen, P. T. and Kilpeläinen, I. A. (2003). Quantitative 2D HSQC (Q-HSQC) via suppression of J-dependence of polarization transfer in NMR spectroscopy: application to wood lignin. J. Am. Chem. Soc. 125, 4362-4367.
Hughes, J. A., Brown, L. R. and Ferro, A. J. (1987). Nucleotide sequence and analysis of the coliphage T3 S-adenosylmethionine hydrolase gene and its surrounding ribonuclease III processing sites. Nucleic Acids Res. 15, 717-729.
Jander, G. and Joshi, V. (2009). Aspartate-derived amino acid biosynthesis in Arabidopsis thaliana. The Arabidopsis Book 7, e0121.
Kim, H. and Ralph, J. (2010). Solution-state 2D NMR of ball-milled plant cell wall gels in DMSO-d(6)/pyridine-d(5). Org. Biomol. Chem. 8, 576-591.
Lee, C., Teng, Q., Zhong, R., Yuan, Y., Haghighat, M. and Ye, Z. H. (2012). Three Arabidopsis DUF579 domain-containing GXM proteins are methyltransferases catalyzing 4-O-methylation of glucuronic acid on xylan. Plant Cell Physiol. 53, 1934-1949.
Li, L., Popko, J. L., Umezawa, T. and Chiang, V. L. (2000). 5-hydroxyconiferyl aldehyde modulates enzymatic methylation for syringyl monolignol formation, a new view of monolignol biosynthesis in angiosperms. J. Biol. Chem. 275, 6537-6545.
Li, L., Hill-Skinner, S., Liu, S., Beuchle, D., Tang, H. M., Yeh, C. T., Nettleton, D. and Schnable, P. S. (2015). The maize brown midrib4 (bm4) gene encodes a functional folylpolyglutamate synthase. Plant J. 81, 493-504.
Mansfield, S. D., Kim, H., Lu, F. and Ralph, J. (2012). Whole plant cell wall characterization using solution-state 2D NMR. Nat. Protoc. 7, 1579-1589.
Mathews, H., Wagoner, W., Kellogg, J. and Bestwick, R. (1995). Genetic transformation of strawberry: stable integration of a gene to control biosynthesis of ethylene. In vitro Cell Dev. Biol. 31, 36-43.
Meyer, K., Shirley, A. M., Cusumano, J. C., Bell-Lelong, D. A. and Chapple, C. (1998). Lignin monomer composition is determined by the expression of a cytochrome P450-dependent monooxygenase in Arabidopsis. Proc. Natl. Acad. Sci. U.S.A. 95, 6619-6623.
Miller, G. (1959). Use of dinitrosalicylic acid reagent for determination of reducing sugar. Anal. Chem. 31, 426-428.
Mottiar, Y., Vanholme, R., Boerjan, W., Ralph, J. and Mansfield, S. D. (2016). Designer lignins: harnessing the plasticity of lignification. Curr. Opin. Biotechnol. 37, 190-200.
Osakabe, K., Tsao, C. C., Li, L., Popko, J. L., Umezawa, T., Carraway, D. T., Smeltzer, R. H., Joshi, C. P. and Chiang, V. L. (1999). Coniferyl aldehyde 5-hydroxylation and methylation direct syringyl lignin biosynthesis in angiosperms. Proc. Natl. Acad. Sci. U.S.A. 96, 8955-8960.
Ralph, J. and Hatfield, R. D. (1991). Pyrolysis-GC-MS characterization of forage materials. J. Agric. Food. Chem. 39, 1426-1437.
Roje, S. (2006). S-Adenosyl-L-methionine: beyond the universal methyl group donor. Phytochemistry 67, 1686-1698.
Ruelland, E., Campalans, A., Selman-Housein, G., Puigdomenech, P. and Rigau, J. (2003). Cellular and subcellular localization of the lignin biosynthetic enzymes caffeic acid-O-methyltransferase, cinnamyl alcohol dehydrogenase and cinnamoyl-coenzyme A reductase in two monocots, sugarcane and maize. Physiol. Plant. 117, 93-99.
Scheller, H. V. and Ulvskov, P. (2010). Hemicelluloses. Annu. Rev. Plant Biol. 61, 263-289.
Shen, B., Li, C. and Tarczynski, M. C. (2002). High free-methionine and decreased lignin content result from a mutation in the Arabidopsis S-adenosyl-L-methionine synthetase 3 gene. Plant J. 29, 371-380.
Sluiter, A., Hames, B., Ruiz, R., Scarlata, C. and Sluiter, J. (2008). Determination of structural carbohydrates and lignin in biomass. In Laboratory Analytical Procedure (Technical Report, NREL/TP-510-42618), Golden, Colo.: National Renewable Energy Laboratory.
Srivastava, A. C., Ramos-Parra, P. A., Bedair, M., Robledo-Hernández, A. L., Tang, Y., Sumner, L. W., Diaz de la Garza, R. I. and Blancaflor, E. B. (2011). The folylpolyglutamate synthetase plastidial isoform is required for postembryonic root development in Arabidopsis. Plant Physiol. 155, 1237-1251.
Srivastava, A. C., Chen, F., Ray, T., Pattathil, S., Peña, M. J., Avci, U., Li, H., Huhman, D. V., Backe, J., Urbanowicz, B., Miller, J. S., Bedair, M., Wyman, C. E., Sumner, L. W., York, W. S., Hahn, M. G., Dixon, R. A., Blancaflor, E. B. and Tang Y. (2015). Loss of function of folylpolyglutamate synthetase 1 reduces lignin content and improves cell wall digestibility in Arabidopsis. Biotechnol. Biofuels 8, 224.
Takano, A., Kakehi, J. and Takahashi, T. (2012). Thermospermine is not a minor polyamine in the plant kingdom. Plant Cell Physiol. 53, 606-616.
Tang, H. M., Liu, S., Hill-Skinner, S., Wu, W., Reed, D., Yeh, C. T., Nettleton, D. and Schnable, P. S. (2014). The maize brown midrib2 (bm2) gene encodes a methylenetetrahydrofolate reductase that contributes to lignin accumulation. Plant J. 77, 380-392.
Tanz, S. K., Castleden, I., Hooper, C. M., Vacher, M., Small, I. and Millar, H. A. (2003). SUBA3: a database for integrating experimentation and prediction to define the SUBcellular location of proteins in Arabidopsis. Nucleic Acids Res. 41(Database issue), D1185-1191.
Urbanowicz, B. R., Peña, M. J., Ratnaparkhe, S., Avci, U., Backe, J., Steet, H. F., Foston, M., Li, H., O'Neill, M. A., Ragauskas, A. J., Darvill, A. G., Wyman, C., Gilbert, H. J. and York, W. S. (2012). 4-O-methylation of glucuronic acid in Arabidopsis glucuronoxylan is catalyzed by a domain of unknown function family 579 protein. Proc. Natl. Acad. Sci. U.S.A. 109, 14253-14258.
Van de Poel, B., Bulens, I., Lagrain, P., Pollet, J., Hertog, M. L., Lammertyn, J., De Proft, M. P., Nicolaï, B. M. and Geeraerd, A. H. (2010). Determination of S-adenosyl-l-methionine in fruits by capillary electrophoresis. Phytochem. Anal. 21, 602-608.
Wagner, A., Tobimatsu, Y., Phillips, L., Flint, H., Torr, K., Donaldson, L., Pears, L. and Ralph, J. (2011). CCoAOMT suppression modifies lignin composition in Pinus radiata. Plant J. 67, 119-129.
Yang, F., Mitra, P., Zhang, L., Prak, L., Verhertbruggen, Y., Kim, J. S., Sun, L., Zheng, K., Tang, K., Auer, M., Scheller, H. V. and Loqué, D. (2013) Engineering secondary cell wall deposition in plants. Plant Biotechnol. J. 11, 325-335.
Yelle, D. J., Ralph, J. and Frihart, C. R. (2008). Characterization of nonderivatized plant cell walls using high-resolution solution-state NMR spectroscopy. Magn. Reson. Chem. 46, 508-517.
Yuan, Y., Teng, Q., Lee, C., Zhong, R. and Ye, Z. H. (2014). Modification of the degree of 4-O-methylation of secondary wall glucuronoxylan. Plant Sci. 219-220, 42-50.
Zhong, R., Morrison, W. H. III., Negrel, J. and Ye, Z. H. (1998). Dual methylation pathways in lignin biosynthesis. Plant Cell 10, 2033-2046.
Ziebell, A., Gracom, K., Katahira, R., Chen, F., Pu, Y., Ragauskas, A., Dixon, R. A. and Davis, M. (2010). Increase in 4-coumaryl alcohol units during lignification in alfalfa (Medicago sativa) alters the extractability and molecular weight of lignin. J. Biol. Chem. 285, 38961-38968.

### Example 2

### Expression of AdoMetase in Poplar

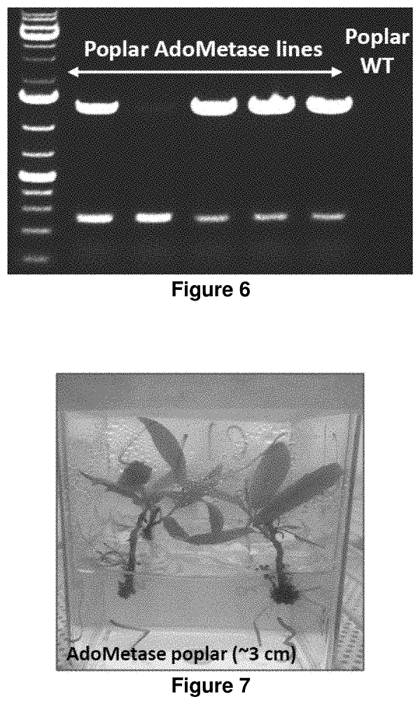

The AdoMetase gene has been transferred to poplar using the method described in Song et al., 2006 (Plant Cell Physiol. 47(11):1582-1589). Four lines are PCR positive for the AdoMetase genes (FIG. 6). Such engineered poplar have grown to at least about 3 cm in height (FIG. 7). Saccharification and lignin measurements are underway. Observation of a dwarf phenotype suggests strong lignin reductions for some lines.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified plant or plant cell comprising a nucleic acid encoding an S-adenosylmethionine hydrolase (AdoMetase) operatively linked to a tissue-specific secondary wall promoter such that there is a specific increased expression of AdoMetase in a secondary cell wall synthesizing tissue when compared secondary cell wall promoter expression compared to corresponding unmodified plant cells.

2. The genetically modified plant or plant cell of claim 1, wherein when grown or cultured there is an increase in H-units and/or β-O-4 linkages formed by the genetically modified plant or plant cell compared to corresponding unmodified plant cells.

3. The genetically modified plant or plant cell of claim 1, wherein the AdoMetase comprises the amino acid sequence of SEQ ID NO:1.

4. The genetically modified plant or plant cell of claim 1, wherein the tissue-specific secondary wall promoter is an IRX1, IRX3, IRX5, IRX8, IRX9, IRX14, IRX7, IRX10, GAUT13, GAUT14, or CESA4 promoter.

5. A method of producing cell wall modified with an increased amount of H-units and/or and β-O-4 linkages, comprising: (a) providing a genetically modified plant of claim 1, (b) growing or culturing the genetically modified plant under conditions in which the AdoMetase is expressed in a secondary cell wall synthesizing tissue, (c) optionally pretreating a biomass formed by the genetically modified plant to form a pretreated biomass, and (d) saccharifying the pretreated biomass to produce one or more sugars.

6. The method of claim 5, wherein the saccharifying step at least about 5% more sugars when compared to the saccharification of biomass grown or cultured from corresponding unmodified plants or plant cells.

7. The method of claim 5, wherein the cell wall of the genetically modified plants or plant cells have at least about 50% more H-units when compared to the cell wall of biomass grown or cultured from corresponding unmodified plants or plant cells.

8. The method of claim 5, wherein the cell wall of the genetically modified plants or plant cells have an about 2.5 to 2.8-fold increase of H-units when compared to the cell wall of biomass grown or cultured from corresponding unmodified plants or plant cells.

9. The method of claim 5, wherein the cell wall of the genetically modified plants or plant cells have at least about 5% fewer S-units when compared to the cell wall of biomass grown or cultured from corresponding unmodified plants or plant cells.

10. The method of claim 5, wherein the cell wall of the genetically modified plants or plant cells have an about 1.4-fold decrease of S-units when compared to the cell wall of biomass grown or cultured from corresponding unmodified plants or plant cells.


SEQUENCE LISTING (5 sequences)

SEQ ID NO: 1 - protein, 150 aa, coliphage T3
MIFTKEPAHVFYVLVSAFRSNLCDEVNMSRHRHMVSTLRAAPGLYGSVESTDLTGCYREAISSAPTEEKTVRVRCKDKAQALNVARLACNEWEQDCVLVYKSQTHTAGLVYAKGIDGYKAERLPGSFQEVPKGAPLQGCFTIDEFGRRWQ

SEQ ID NO: 2 - DNA, 21 bp, Artificial Sequence
  Forward primer for Tub8
gggctaaaggacactacactg

SEQ ID NO: 3 - DNA, 25 bp, Artificial Sequence
  Reverse primer for Tub8
cctcctgcacttccacttcgtcttc

SEQ ID NO: 4 - DNA, 26 bp, Artificial Sequence
  Forward primer for AdoMetase
ccaatctttgcgatgaggttaatatg

SEQ ID NO: 5 - DNA, 23 bp, Artificial Sequence
  Reverse primer for AdoMetase
gtcctgaacttgccaccttcttc